(19) United States
(12) Patent Application Publication
(10) Pub. No.: US 2023/0404474 A1
(43) Pub. Date: Dec. 21, 2023

(54) EVALUATION METHOD OF SLEEP QUALITY AND COMPUTING APPARATUS RELATED TO SLEEP QUALITY

(71) Applicants: Wistron Corporation, New Taipei City (TW); Taipei Medical University, Taipei City (TW)

(72) Inventors: Yu-Hsuan Ho, New Taipei City (TW); Yu-Wen Huang, New Taipei City (TW); Wen-Te Liu, New Taipei City (TW)

(73) Assignees: Wistron Corporation, New Taipei City (TW); Taipei Medical University, Taipei City (TW)

(21) Appl. No.: 18/190,125

(22) Filed: Mar. 27, 2023

Related U.S. Application Data
(60) Provisional application No. 63/352,644, filed on Jun. 16, 2022.

(30) Foreign Application Priority Data
Oct. 3, 2022 (TW) 111137595
Jan. 16, 2023 (TW) 112101793

(51) Int. Cl. A61B 5/00

(52) U.S. Cl. CPC A61B 5/4806; A61B 5/7235

(57) ABSTRACT

An evaluation method of sleep quality and a computing apparatus related to sleep quality are provided. In the evaluation method, sensing data is obtained. The sensing data is generated based on a radar echo. The sensing data is transformed into feature data. The feature data includes a statistic of a plurality of feature points on a waveform of the radar echo. Sleep quality information is determined according to the feature data. Accordingly, sleep quality may be evaluated through non-touch sensing.

### CROSS-REFERENCE TO RELATED APPLICATION

This application claims the priority benefits of U.S. provisional application Ser. No. 63/352,644, filed on Jun. 16, 2022, Taiwan application serial no. 111137595, filed on Oct. 3, 2022, and Taiwan application serial no. 112101793, filed on Jan. 16, 2023. The entirety of each of the above-mentioned patent applications is hereby incorporated by reference herein and made a part of this specification.

### BACKGROUND OF THE INVENTION

### Field of the Invention

The invention relates to a data analysis technique, and in particular relates to an evaluation method of sleep quality and a computing apparatus related to sleep quality.

### Description of Related Art

Sleep apnea refers to the symptoms of involuntary weakening or even cessation of breathing during sleep. The cessation of breathing is often unnoticed until the body is severely deprived of oxygen and wakes up due to discomfort. However, lack of oxygen may harm the body, and the patient may even die suddenly from cardiovascular disease. People with sleep apnea are often unaware of symptoms. Symptoms may only be discovered when the patient goes to the hospital for detection and diagnosis with special equipment.

### SUMMARY OF THE INVENTION

Accordingly, an embodiment of the invention provides an evaluation method of sleep quality and a computing apparatus related to sleep quality that may readily detect sleep quality.

An evaluation method of sleep quality of an embodiment of the invention includes (but not limited to) the following steps. Sensing data is obtained. The sensing data is generated based on a radar echo. The sensing data is transformed into feature data. The feature data includes a statistic of a plurality of feature points on a waveform of the radar echo. Sleep quality information is determined according to the feature data. The sleep quality information is related to whether the sleep quality is good or bad.

A computing apparatus related to sleep quality of an embodiment of the invention includes (but not limited to) a memory and a processor. The memory is configured to store a program code. The processor is coupled to the memory. The processor loads the program code to execute: obtaining sensing data, transforming the sensing data into feature data, and determining sleep quality information according to the feature data. The sensing data is generated based on a radar echo. The feature data includes a statistic of a plurality of feature points on a waveform of the radar echo. The sleep quality information is related to whether the sleep quality is good or bad.

Based on the above, according to the evaluation method of sleep quality and the computing apparatus related sleep quality of the embodiments of the invention, the sleep quality information is predicted by using radar-based sensing data. The feature data obtained from the sensing data corresponds to polysomnography (PSG). PSG may be reflected in respiratory events, and the respiratory events are related to the degree of sleep quality. Accordingly, sleep quality may be evaluated through non-touch sensing.

In order to make the aforementioned features and advantages of the disclosure more comprehensible, embodiments accompanied with figures are described in detail below.

### BRIEF DESCRIPTION OF THE DRAWINGS

FIG. 1 is a device block diagram of a computing apparatus and a radar according to an embodiment of the invention.

FIG. 2 is a flowchart of an evaluation method of sleep quality according to an embodiment of the invention.

FIG. 3 is a schematic diagram of waveform-related feature data according to an embodiment of the invention.

FIG. 4 is a schematic diagram of trend-related feature data according to an embodiment of the invention.

FIG. 5 is a schematic diagram of a Deep Neural Decision Tree (DNDT) according to an embodiment of the invention.

FIG. 6 is a schematic diagram of sensing data and events according to an embodiment of the invention.

FIG. 7 is a schematic diagram of indicator verification according to an embodiment of the invention.

### DESCRIPTION OF THE EMBODIMENTS

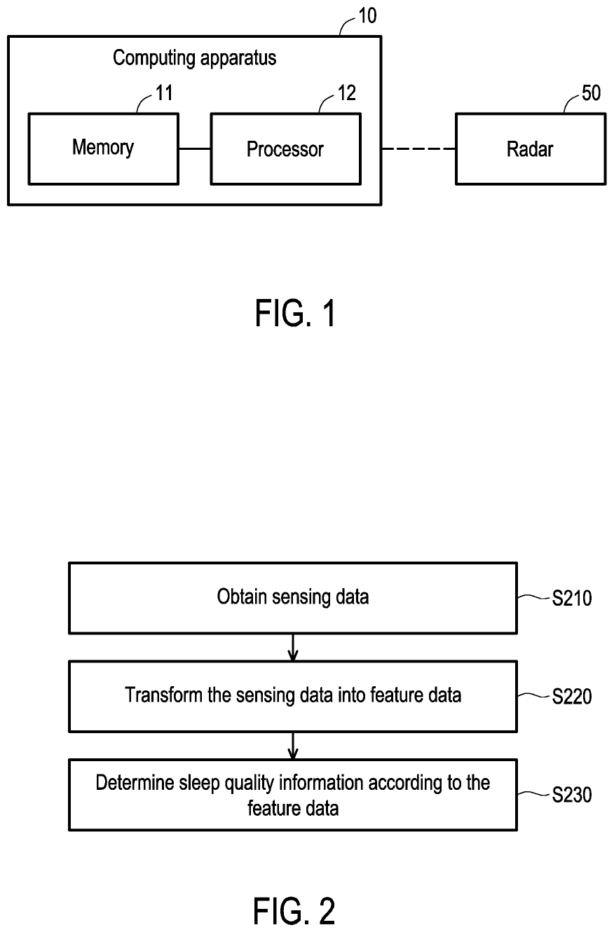

FIG. 1 is a device block diagram of a computing apparatus 10 and a radar 50 according to an embodiment of the invention. Please refer to FIG. 1, the computing apparatus 10 includes (but not limited to) a memory 11 and a processor 12. The computing apparatus 10 may be a mobile phone, a tablet computer, a notebook computer, a desktop computer, a voice assistant apparatus, a smart home appliance, a wearable apparatus, a vehicle-mounted apparatus, or other electronic apparatuses.

The memory 11 may be any form of a fixed or movable random-access memory (RAM), read-only memory (ROM), flash memory, traditional hard disk drive (HDD), solid-state drive (SSD), or similar devices. In an embodiment, the memory 11 is configured to store a program code, a software module, a configuration, data, or a file (for example, data, an event, information, a model, or a feature), and is described in detail in subsequent embodiments. The processor 12 is coupled to the memory 11. The processor 12 may be a

central processing unit (CPU), a graphics processing unit (GPU), or other programmable general-purpose or special-purpose microprocessors, digital signal processors (DSPs), programmable controllers, field-programmable gate arrays (FPGAs), application-specific integrated circuits (ASICs), neural network accelerators, or other similar devices or a combination of the above devices. In an embodiment, the processor 12 is configured to perform all or part of the operations of the computing apparatus 10, and may load and execute each of the program codes, software modules, files, and data stored in the memory 11. In some embodiments, some operations in a method of an embodiment of the invention may be implemented by different or the same processor 12. In an embodiment, the processor 12 is connected to the radar 50. For example,

the radar 50 is connected to the processor 12 via USB, Thunderbolt, Wi-Fi, Bluetooth, or other wired or wireless communication techniques. For another example, the computing apparatus 10 has a built-in radar 50, and the processor 12 is connected to the radar 50 through an internal circuit. The radar 50 may be a frequency-modulated continuous wave (FMCW) radar or an impulse radio (IR)-ultra-wideband (UWB) radar. In an embodiment, the radar 50 is configured to generate sensing data. The sensing data is generated based on a radar echo. The radar echo refers to an echo signal reflected by the transmitted signal of the radar 50 by an object (e.g., human body or clothes). The sensing data is the sensing result of the radar 50. Examples include in-phase and/or quadrature signals.

In an embodiment, the frequency of the transmitted signal of the radar 50 may be 24 GHz or other frequencies that may reflect the human body (e.g., chest or abdomen).

In an application scenario, the radar 50 may be placed at the head of the bed, beside the bed, or at the end of the bed, and the radar 50 transmits a signal towards the chest or abdomen of the human body, and accordingly detects the rise and fall of the chest or abdomen. However, the location and orientation of the radar 50 may still be changed according to actual needs, and are not limited by the embodiments of the invention.

Hereinafter, the method described in an embodiment of the invention is described with various apparatuses, devices, and modules in the computing apparatus 10 and the radar 50. Each of the processes of the present method may be adjusted according to embodiment conditions and is not limited thereto.

FIG. 2 is a flowchart of an evaluation method of sleep quality according to an embodiment of the invention. Referring to FIG. 2, the processor 12 obtains sensing data (step S210). Specifically, the sensing data is generated based on a radar echo. For example, the radar 50 emits a continuous wave signal, and the continuous wave signal is reflected by the chest or abdomen to form a radar echo. The radar 50 may receive the radar echo and generate the sensing data accordingly. The sensing data takes in-phase (I) and quadrature (Q) signals as an example, an in-phase signal I1 is [0.164144 0.179153 0.194716 . . . 1.600188 1.590467 1.586891], and a quadrature signal Q1 is [2.295545 2.278471 2.270613 . . . 1.031502 1.027573 1.049331].

In an embodiment, the processor 12 may accumulate the sensing data for a period of time. This period of time is, for example, 1, 5, or 8 hours.

The processor 12 transforms the sensing data into feature data (step S220). In an embodiment, the feature data includes the variance between two channels or within a single channel in the sensing data. These two channels may be in-phase and quadrature signals. The mathematical expression of the variance is:

Cov(X,Y)=E((X−μ_{X})(Y−μ_{Y})) (1)

Cov is the variance, X and Y are either in-phase or quadrature signals, μ_{X} is the average value of X, and μ_{Y} is the average value of Y. Taking the above in-phase signal I1 and quadrature signal Q1 as examples, the variance thereof is −0.21645484961728612.

In an embodiment, the feature data includes entropy of the sensing data. In information theory, entropy refers to the average amount of information contained in each received message, which is a measure of uncertainty, and the entropy is increased as the source of information becomes more random. The entropy-based feature is, for example, relative entropy, conditional entropy, mutual information, information entropy, Shannon entropy, or block entropy.

Taking Shannon entropy as an example, the entropy H of a random variable X (with a range of x={ x₁, . . . , xₙ}) is defined as:

−ΣₓP_{X}(x)log_{b}P_{X}(x) (2),

P_{X} is the probability mass function of the random variable X, and b is the base used for the logarithm. Taking the above in-phase signal I1 and quadrature signal Q1 as examples, the conditional entropy thereof is 1.4112874013149717.

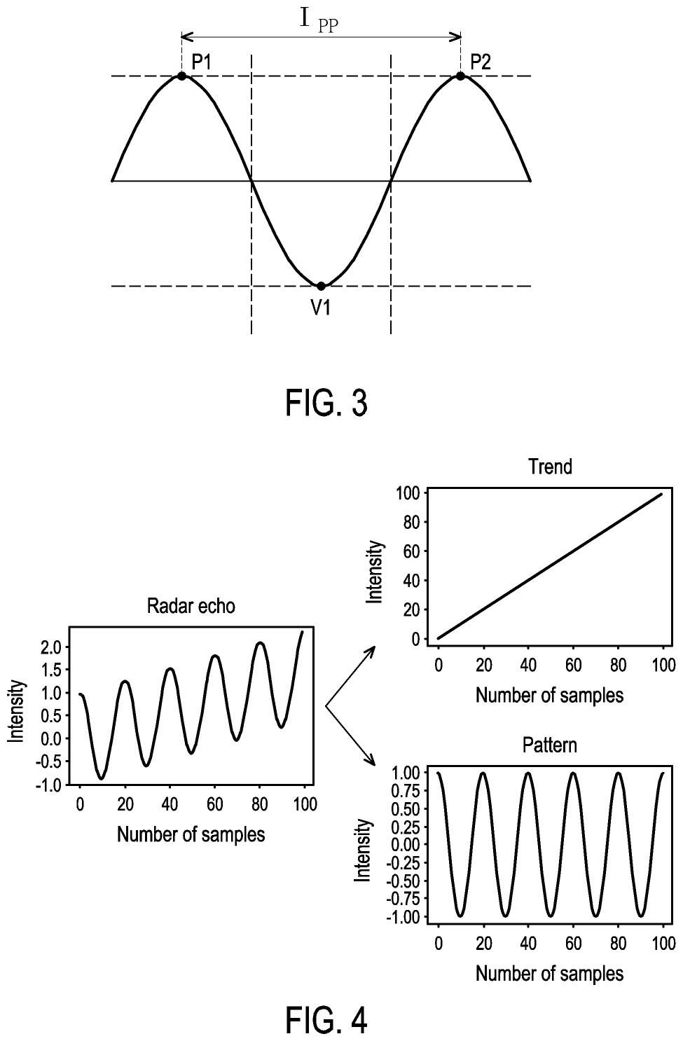

In an embodiment, the feature data includes a statistic of a plurality of feature points on a waveform of the radar echo. The feature points may be peak values and/or valley values in the waveform. For example, FIG. 3 is a schematic diagram of waveform-related feature data according to an embodiment of the invention. Please refer to FIG. 3, the waveform includes peak values P1 and P2 and a valley value V1. The peak values P1 and P2 may be the maximum values within one or a plurality of periods. The valley value V1 may be the minimum value in one or a plurality of periods.

In addition, the statistic may be the interval between two feature points, the variation of the interval, and/or the total number of those feature points. Taking FIG. 3 as an example, the statistic is an interval I_{PP} between two peak values P1. However, the statistic may also be the interval between the valley value V1 and another adjacent valley value (not shown), the interval between the peak value P1/P2 and the valley value V1, or the interval between two specified points in the waveform. The variation of an interval is, for example, the difference between two or more intervals, such as the difference between the interval I_{PP} and another interval I_{PP} (not shown, such as the interval between the peak value P2 and the next peak value). The total number of feature points is, for example, the total number of peak values and/or valley values within a period of time (e.g., 1000 sampling points or 3 hours).

In an embodiment, the processor 12 may separately determine the statistics of the waveforms of the in-phase and quadrature signals, and may also take the average value of the statistics of two signals as the feature data.

In an embodiment, the feature data includes the trend of the waveform, and the trend is the intensity variation of the waveform without pattern characteristic. A pattern characteristic may be a periodic variation of a waveform. For example, the sine wave signal is increased from zero to the maximum value, decreased from the maximum value to the minimum value, and then increased from the minimum value to zero repeatedly. After the pattern characteristic is removed, the trend of the waveform is left, i.e., intensity variation. The processor 12 abstracts the trend (that is, eliminates the interference of the absolute signal intensity), which may be used as feature data describing sleep quality (for example, a respiratory event or a sleep event).

For example, FIG. 4 is a schematic diagram of trend-related feature data according to an embodiment of the invention. Please refer to FIG. 4, a radar echo may be divided into a trend and a pattern. The trend is a linear function, and the slope of the linear function is positive, so the trend of this waveform is gradually increased in intensity. The pattern is a sine wave. In another embodiment, the trend may also be a curve. In addition, taking a programming language and the above in-phase signal I1 as an example, the python package: seasonal_decompose may divide the maximum value by the minimum value in the trend of the in-phase signal I1, to obtain: 1.207502488 (as a representative value of the trend).

In an embodiment, the processor 12 may separately determine the trend of the waveforms of the in-phase and quadrature signals, and may also take the average value of the trend of the two signals as the feature data.

In an embodiment, the processor 12 may select one or more of the above statistics, variance, entropy, and trend of the sensing data as the feature data.

Referring to FIG. 2, the processor 12 determines sleep quality information according to the feature data (step S230). Specifically, the sleep quality information is related to whether the sleep quality is good or bad. In an embodiment, the sleep quality information includes a respiratory event, such as normal breathing, hypopnea, flow limitation, obstructed breathing, awake, or an apnea event. Apnea is defined as a complete cessation of breathing during sleep, in which breathing is temporarily stopped and airflow is interrupted for at least 10 seconds, which is referred to as one event (i.e., one sleep apnea). Hypopnea breathing is defined as an abnormal breathing pattern, which for adults is at least more than 10 seconds at a time. The airflow and respiratory movement of the chest and abdomen are reduced to only 30% to 50% of the normal situation, and at the same time, oxygen saturation in the blood is reduced by at least 4%. Flow limitation is defined as an abnormal breathing pattern in which the flow rate of airflow is lower than normal due to partial obstruction of the airway.

The processor 12 may predict the respiratory event according to the feature data. The feature data of an embodiment of the invention includes features obtained by comparing multiple polysomnography (PSG) tests (for example, respiratory airflow, chest movement, abdominal muscle behavior, or EEG) that may better distinguish respiratory events. However, different from recognizing the respiratory event based on

PSG, an embodiment of the invention recognizes the respiratory event based on the feature data of the radar.

In an embodiment, the processor 12 may predict the respiratory event through a machine learning model. A machine learning model is trained to understand the correlation between the feature data and the respiratory event. The machine learning model is, for example, based on deep neural decision tree (DNDT), deep learning neural network, decision tree, random forest, or other machine learning algorithms. The deep learning neural network is, for example, a temporal convolutional network (TCN) and a convolutional neural network (CNN). DNDT is a hybrid deep learning and decision tree strategy. The machine learning algorithm may analyze training samples to obtain patterns therefrom, so as to predict unknown data via the patterns. For example, the machine learning model establishes the correlation between the nodes in a hidden layer between the feature data (i.e., the input of the model) and the respiratory event (i.e., the output of the model) according to labeled samples (e.g., feature data of known hypopnea events, or feature data of known normal breathing events). The machine learning model is a model constructed after learning, and may accordingly infer data to be evaluated (for example, the feature data).

For example, CNN may learn image-related features, and TCN may learn temporal features. DNDT combines the concepts of decision trees and deep learning from the field of machine learning. Traditional decision trees may not optimize each tree node, thus causing limitations in judgment. However, DNDT allows each node of the decision tree to be weighted by a learning machine undergoing deep learning.

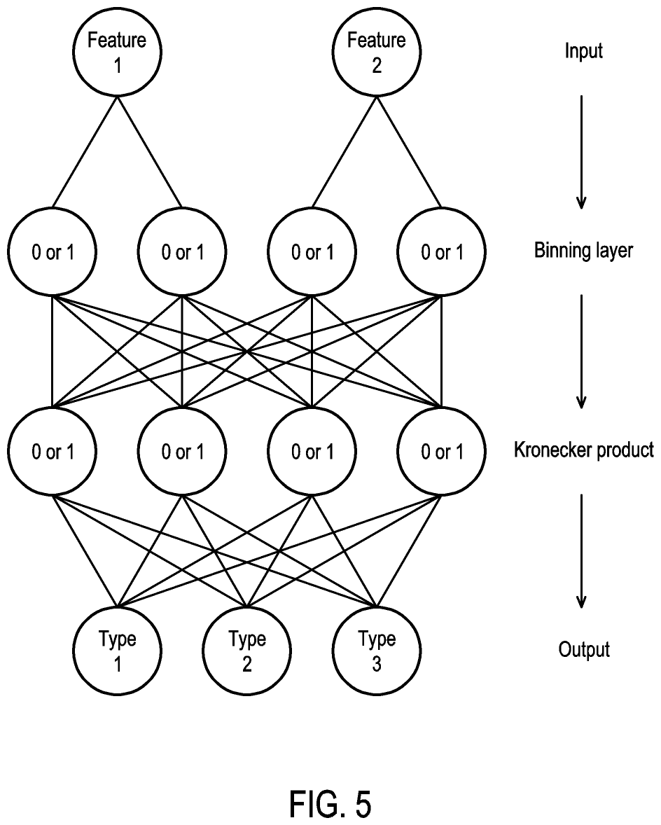

For example, FIG. 5 is a schematic diagram of a DNDT according to an embodiment of the invention. Please refer to FIG. 5, during the training process of the model, through deep learning, the weight of each node in the decision tree is continuously changed (such as the weight between an input and a binning layer or the weight between a Kronecker product and an output) to further reduce loss value, in order to achieve the object of training weights. The binning layer is configured to implement a differentiable clustering function, and the Kronecker product is configured to decide which leaf value in the decision tree to predict. Therefore, learning the feature data through the architecture of DNDT may be further used to determine whether a normal sleep or a sleep/breathing event occurs.

Taking the above in-phase signal I1 as an example, the processor 12 may directly transform the in-phase signal I1 into a two-dimensional matrix (for example, the matrix size is 40×50 or 30×50) (as the input of the model) to learn the machine learning model. Alternatively, the form of the feature data may be varied according to different time scales (for example, 100, 1500, 2000, or 2500 sampling points, but not limited thereto). In some application scenarios, the longer the time or the more sampling points, the higher the accuracy, but not limited thereto. Alternatively, the processor 12 may use table-type feature data (as the input of the model) to learn the machine learning model. For example, the feature data of these time series is transformed into values and then organized into the following table:

TABLE 1

| | | Variance | | Peak | |
|---|---|---|---|---|---|
| | | between in- | | value- | |
| Variance of | Variance of | phase and | | to-peak | |
| in-phase | quadrature | quadrature | | value | |
| channel | channel | channels | Entropy | interval | Trend |
| 0.058919 | 0.128993 | 0.033093 | 0.58167 | 0.052821 | 1.052752 |
| 0.049791 | 0.099063 | 0.02131 | 0.478866 | 0.011539 | 1.043053 |
| 0.013008 | 0.097488 | −0.00206 | 0.622213 | 0.006285 | 1.01288 |
| 0.010046 | 0.114137 | −0.008 | 0.669016 | 0.000769 | 1.007484 |
| 0.046572 | 0.138222 | 0.018872 | 0.648689 | 0.024544 | 1.044307 |

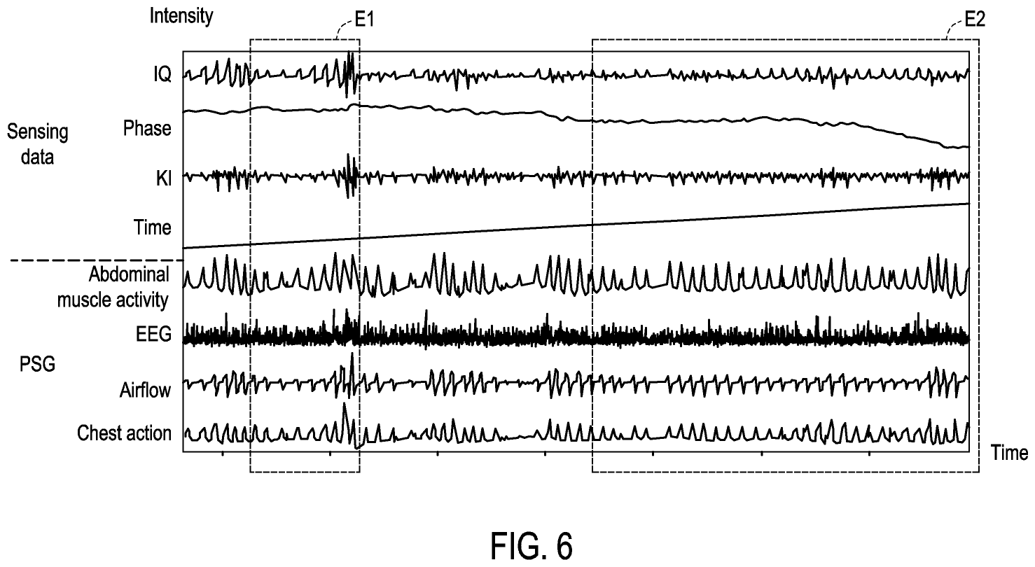

FIG. 6 is a schematic diagram of sensing data and events according to an embodiment of the invention. Please refer to FIG. 6, an event E1 is a hypopnea event, and the sensing data and PSG may reflect obvious rapid fluctuations. For example, the last segment of the event E1 has a higher or lower value. An event E2 is a normal sleep event, so the waveforms of the sensing data and PSG are generally changed regularly.

The processor 12 may count the number and/or duration of specific respiratory events within a period of time (e.g., 2, 5, or 8 hours) as sleep quality information. The higher the statistic of the normal sleep event, the better the sleep quality (for example, the degree of quality is higher, and high represents excellent); the higher the statistic such as hypopnea and/or apnea, the worse the sleep quality (the degree of quality is lower, and low means bad).

In an embodiment, the sleep quality information includes a sleep statistical indicator. The sleep statistical indicator is a respiratory disturbance index (RDI) or an apnea-hypopnea index (AHI). RDI is the number of interrupted breathing during sleep, and some people use AHI directly. Under the same measurement, the RDI index is slightly larger than the AHI index. According to the standards of the American Sleep Association, an AHI of less than 5 is normal, an AHI of 5 to 14 is mild, an AHI of 15 to 29 is moderate, and an AHI of 30 or more is severe respiratory disturbance. That is to say, the lower the sleep statistical indicator, the higher the degree of sleep quality, and high represents good; the higher the sleep statistical indicator, the lower the degree of sleep quality, and low represents poor.

In an embodiment, the processor 12 may determine the sleep statistical indicator according to a predicted respiratory event. The processor 12 may count the prediction results (e.g., the output of the machine learning model) of previous respiratory events within a period of time (e.g., 3 hours, 5 hours, or 8 hours), and generate a predicted sleep statistical indicator, i.e., a value obtained by dividing the number of specific respiratory events by the statistical time.

For example, Table (2) is the corresponding relationship between time points (for example, every minute, every 30 minutes, or every hour) and predicted results:

| | Prediction result |
|---|---|
| Time point | of respiratory event |
| 1 | 0 |
| 2 | 1 |
| 3 | 1 |
| 4 | 0 |
| . . . | 0 |
| . . . | . . . |
| N (positive integer) | 0 |

wherein “0” means no event and “1” means event. The AHI may be obtained by dividing the number of hypopnea events by the statistical time. That is, how often 1 occurs per unit time. In addition, the prediction results of each “1” may be compared and verified with PSG to improve accuracy.

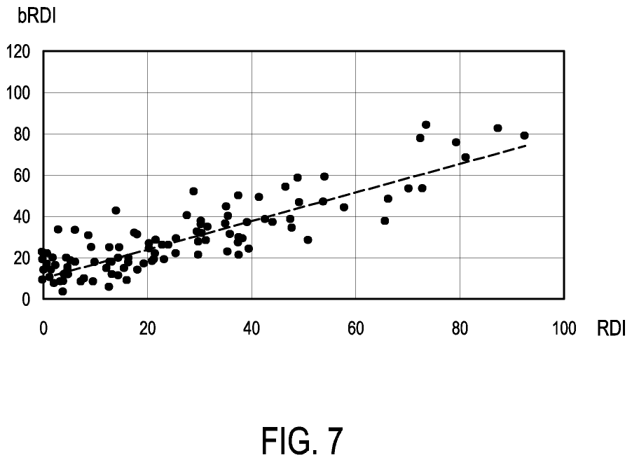

In order to verify whether an RDI type value (that is, the sleep statistical indicator) produced by an embodiment of the invention may be close to the real RDI value, the data of 103 people in a clinical research case were actually collected for sleep testing in a sleep center, and these data were used for verification. FIG. 7 is a schematic diagram of indicator verification according to an embodiment of the invention. Please refer to FIG. 7, trend and accuracy analysis (Table (3)) was performed with RDI type values (expressed with the bRDI of Y axis) drawn in an embodiment of the invention and the RDI (with the RDI of X axis) determined by a sleep technician. It may be seen from FIG. 7 that the RDI type values obtained in an embodiment of the invention are positively correlated with the real RDI value, and the correlation degree (for example, 0.7481) is greater than 0.7.

In addition, clinically, RDI greater than or equal to 15/hours (h) and greater than or equal to 30/h are defined as having moderate and severe symptoms of apnea, and the comparison results may be obtained in Table (3):

TABLE 3

| | Set RDI | Set RDI |
|---|---|---|
| | greater than or | greater than or |
| | equal to 15/h | equal to 30/h |
| Hit rate | as positive | as positive |
| True positive | 49/64 (76.56%) | 36/40 (90.00%) |
| True negative | 32/39 (82.05%) | 51/63 (80.95%) |
| Correlation between | 0.7481 | 0.7481 |
| bRDI and actual RDI | | |

True positive is the proportion determined to be positive by an embodiment of the invention and is actually positive, and true negative is the proportion determined to be negative by an embodiment of the invention and is actually negative. It may be known that the proportion of correct positives (e.g., RDI greater than 15 per hour or 30 per hour) is greater than 75% and the proportion of correct negatives (e.g., RDI of less than 15 per hour or 30 per hour) is greater than 80%.

In an embodiment, the processor 12 may predict the sleep statistical indicator according to the feature data. For example, the processor 12 additionally trains another machine learning model to accordingly understand the correlation between the feature data and the predicted sleep statistical indicator. For the introduction of the machine learning model, reference may be made to the above description, and details are not repeated herein. For example, the machine learning model establishes the correlation between the nodes in a hidden layer between the feature data (i.e., the input of the model) and the sleep statistical indicator (i.e., the output of the model) according to labeled samples (e.g., known feature data for RDI, or known feature data of AHI). Since the feature data of an embodiment of the invention may be configured to distinguish a respiratory event and the sleep statistical indicator is obtained based on the respiratory event (for example, the number of specific one or more respiratory events divided by the statistical time), it may thus be demonstrated that the feature data may be configured to predict the sleep statistical indicator.

Based on the above, in the evaluation method of sleep quality and the computing apparatus related to sleep quality of the embodiments of the invention, the sleep quality is determined according to the feature data transformed from the radar sensing data (for example, related to variance, entropy, waveform, and/or trend). In this way, the sleep quality may be evaluated in a non-contact sensing manner.

Although the invention has been described with reference to the above embodiments, it will be apparent to one of ordinary skill in the art that modifications to the described embodiments may be made without departing from the spirit of the disclosure. Accordingly, the scope of the disclosure is defined by the attached claims not by the above detailed descriptions.

What is claimed is:

1. An evaluation method of sleep quality, comprising:
obtaining sensing data, wherein the sensing data is generated based on a radar echo;
transforming the sensing data into feature data, wherein the feature data comprises a statistic of a plurality of feature points of the radar echo on a waveform; and
determining sleep quality information according to the feature data, wherein the sleep quality information is related to a degree of sleep quality.

2. The evaluation method of sleep quality of claim 1, wherein the feature points comprise at least one of a peak value and a valley value, and the statistic comprises at least one of an interval between two of the feature points, a variation of the interval, and a total number of the feature points.

3. The evaluation method of sleep quality of claim 1, wherein the feature data further comprises a variance between two channels or within a single channel in the sensing data.

4. The evaluation method of sleep quality of claim 1, wherein the feature data further comprises an entropy of the sensing data.

5. The evaluation method of sleep quality of claim 1, wherein the feature data further comprises a trend of the waveform, and the trend is an intensity variation of the waveform without a pattern characteristic.

6. The evaluation method of sleep quality of claim 1, wherein the sleep quality information comprises a respiratory event, and the step of determining the sleep quality information according to the feature data comprises:
predicting the respiratory event according to the feature data.

7. The evaluation method of sleep quality of claim 6, wherein the step of predicting the respiratory event according to the feature data comprises:
predicting the respiratory event by a machine learning model, wherein the machine learning model is trained to understand a correlation between the feature data and the respiratory event.

8. The evaluation method of sleep quality of claim 7, wherein the machine learning model is based on one of a deep neural decision tree, a deep learning neural network, and a decision tree, and the respiratory event is a normal breathing, a hypopnea, a flow limitation, an obstructed breathing, an awake, or an apnea event.

9. The evaluation method of sleep quality of claim 6, wherein the sleep quality information further comprises a sleep statistical indicator, the sleep statistical indicator is a respiratory disturbance index or an apnea-hypopnea index, and the step of predicting the respiratory event according to the feature data comprises:
determining the sleep statistical indicator according to the predicted respiratory event.

10. The evaluation method of sleep quality of claim 1, wherein the sleep quality information comprises a sleep statistical indicator, the sleep statistical indicator is a respiratory disturbance index or an apnea-hypopnea index, and the step of determining the sleep quality information according to the feature data comprises:
predicting the sleep statistical indicator according to the feature data.

11. A computing apparatus related to sleep quality, comprising:
a memory storing a program code; and
a processor coupled to the memory and loading the program code to execute:
obtaining sensing data, wherein the sensing data is generated based on a radar echo;
transforming the sensing data into feature data, wherein the feature data comprises a statistic of a plurality of feature points of the radar echo on a waveform; and
determining sleep quality information according to the feature data, wherein the sleep quality information is related to a degree of sleep quality.

12. The computing apparatus related to sleep quality of claim 11, wherein the feature points comprise at least one of a peak value and a valley value, and the statistic comprises at least one of an interval between two of the feature points, a variation of the interval, and a total number of the feature points.

13. The computing apparatus related to sleep quality of claim 11, wherein the feature data further comprises a variance between two channels or within a single channel in the sensing data.

14. The computing apparatus related to sleep quality of claim 11, wherein the feature data further comprises an entropy of the sensing data.

15. The computing apparatus related to sleep quality of claim 11, wherein the feature data further comprises a trend of the waveform, and the trend is an intensity variation of the waveform without a pattern characteristic.

16. The computing apparatus related to sleep quality of claim 11, wherein the sleep quality information comprises a respiratory event, and the processor further executes:
predicting the respiratory event according to the feature data.

17. The computing apparatus related to sleep quality of claim 16, wherein the processor further executes:
predicting the respiratory event by a machine learning model, wherein the machine learning model is trained to understand a correlation between the feature data and the respiratory event.

18. The computing apparatus related to sleep quality of claim 17, wherein the machine learning model is based on one of a deep neural decision tree, a deep learning neural network, and a decision tree, and the respiratory event is a normal breathing, a hypopnea, a flow limitation, an obstructed breathing, an awake, or an apnea event.

19. The computing apparatus related to sleep quality of claim 16, wherein the sleep quality information further comprises a sleep statistical indicator, the sleep statistical indicator is a respiratory disturbance index or an apnea-hypopnea index, and the processor further executes:
determining the sleep statistical indicator according to the predicted respiratory event.

20. The computing apparatus related to sleep quality of claim 11, wherein the sleep quality information comprises a sleep statistical indicator, the sleep statistical indicator is a respiratory disturbance index or an apnea-hypopnea index, and the processor further executes:
predicting the sleep statistical indicator according to the feature data.
